(19) United States
(12) Patent Application Publication
(10) Pub. No.: US 2001/0039048 A1
(43) Pub. Date: Nov. 8, 2001

(54) Recombinant adenovirus and its use for the prevention and treatment of fibrotic disease

(75) Inventors: Chu-Tse Wu, Beijing (CN); Xiaoqin Ha, Beijing (CN); Yuanmin Li, Beijing (CN)

Correspondence Address: Ladas & Parry, 26 West 61st Street, New York, NY, 10023, US

(73) Assignee: BAIHUAN BIOMEDICAL RESEARCH CENTER (BBRC) ACADEMY

(21) Appl. No.: 09/775,534

(22) Filed: Feb. 1, 2001

(30) Foreign Application Priority Data
Feb. 2, 2000 (CN) 00100739.4

(51) Int. Cl. C12N 7/01; C12N 7/00; A61K 39/23; C07K 1/00

(52) U.S. Cl. 435/325; 435/235.1; 530/350; 435/320.1; 424/233.1

(57) ABSTRACT

This invention is involved in the biomedical field, particularly it is involved in a kind of recombinant adenovirus carrying human hepatocyte growth factor gene and its application in prevention and treatment of fibrotic diseases and vascular obliterative diseases. The reffered recombinant adenovirus carrying human hepatocyte growth factor gene is obtained by transduction of the whole length cDNA of the domain encoding human hepatocyte growth factor gene into a replication-defective recombinant adenovirus GT4050 as the vector. By injection of the referred recombinant adenovirus into the caudal vein of rat, the curative effect for rat liver fibrosis is achieved, and by smearing of the referred recombinant adenovirus on wound surface on the ear of rabbit the formation of scar can be prevented.

This invention is involved in the biomedical field, particularly it is involved in a recombinant adenovirus carrying human hepatocyte growth factor gene and its application in prevention and treatment of fibrotic diseases and vascular obliterative diseases.

Fibrotic diseases, such as liver fibrosis, pulmonary fibrosis, renal fibrosis, and excessive scarring, are one kind of the diseases affecting severely the human life and health. Liver fibrosis is one of the major pathologic features of chronic liver diseases and is also one of the important causes leading to further development of chronic viral hepatitis, metabolic disorder, chronic alcoholism, and others. At present, treatment of liver fibrosis is still staying on the level of delaying or blocking the process of fibrosis, but no effective means can be adopted to reverse the fibrotic process so as to achieve the curative effect for it. Although there are reports on treatment of liver cirrhosis by using plasmids⁽¹⁾, however, their extensive clinical use would be limited by shortcomings such as administration with a large amount and repeated administrations are required. Up to now treatment of liver fibrosis with recombinant adenovirus has not been reported.

Formation of scar is the product and a symbol of wound healing. However, excessive proliferation of scar is a morbid manifestation and is a major complication in trauma surgery. Scars may affect body appearance and interfere with the function of the organ, and what is more, they can transform into cancers. At present the main treatments for scar proliferation are surgical operation, compression, cryotherapy, and so on, which are all far from ideal. Hence, prevention from scar formation is of important significance. Up to now there is no report on prevention of scars by external application of bioengineering products in the literature.

In view of the important roles played by cytokines in fibrotic diseases, studies on relationship between cytokines and formation of scars are particularly concerned. The hepatocyte growth factor (HGF) was considered at first as a mitogen for hepatocytes, however, it was found later that HGF regulates division, migration, and morphogenesis of a variety of cells. Especially, it plays an important role in development and regeneration of hepatocytes. As a cytokine derived from mesenchymal cells, HGF can inhibit remarkably the synthesis and secretion of transforming growth factor (TGF β) and elevate the activity of collagenase , but it does nothing in stimulation of fibroblasts. Thus it effectively lowers the TGFβ level and degradates collagen fiber in the tissue, both of which are important in prevention and treatment of fibrotic diseases.

Recombinant adenovirus has drawn much attention to by its high transfection efficiency of genes into various kinds of cell and has become an important gene vector system, which is developing rapidly and is used in clinical practice of gene therapy. Owing to adenovirus administered into the body are mostly focused in the liver, therefore, it becomes an ideal vector of genes taking liver as the target organ. When adenovirus are applied topically on wounds, their transfection efficiency is high and is superior to that applied via gene gun.

In summary, up to now there is no effective means for prevention and treatment of fibrotic diseases, which remain as one of the hardnut to be cracked in modern medicine.

The first purpose of this invention lies in that construction of a kind of recombinant adenovirus carrying human hepatocyte growth factor gene.

The second purpose of this invention lies in that the recombinant adenovirus carrying human hepatocyte growth factor gene is used , with certain titers, for preparation of injections, smear solutions, sprays, and other dosage forms for the sake of prevention and treatment of fibrotic diseases and vascular obliterative diseases in clinical practice.

The one preferred embodiment of this invention is as follows:

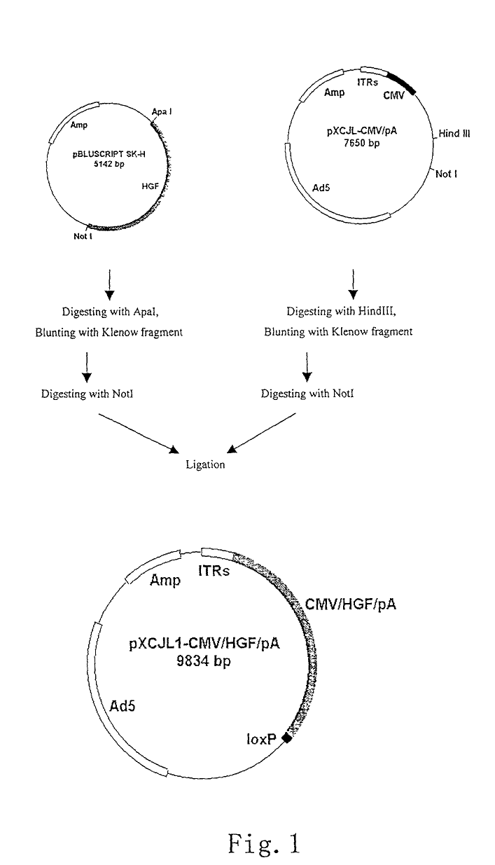

1. The whole length cDNA of domain encoding human hepatocyte growth factor gene was isolated from the cDNA library of human fetal liver and it was cloned in BamHI and SalI sites of the pBLUSCRIPT vector; then HGF cDNA was cleaved from ApaI and NotI sites of pBLUSCRIPT-HGF and was cloned to HindIII and NotI sites of pXCJL1-CMV/pA vector, which contains the CMV promotor, polyA signal and the left side sequence of the adenoviral genome, hence the expression box carrying human hepatocyte growth factor gene and the shuttle plasmid of the left side sequence of adenoviral genome(pXCJL1-CMV/pA-HGF) were obtained (See FIG. 1).

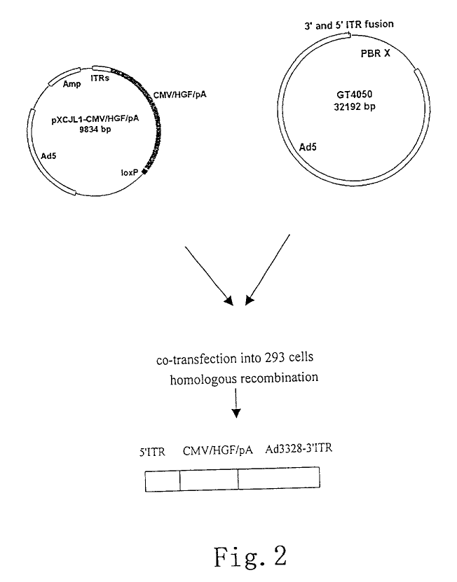

2. The shuttle plasmid pXCJL1-CMV/pA-HGF was co-transfected into 293 cells (human fetal kidney cell line transformed by adenoviral E1 gene) along with a plasmid GT4050 which contained the most rightward sequences of human type 5 adenovirus genome with a partial deletion (E1 and E3 domains and packaging signal). The recombinant replication-deficient adenovirus carrying human hepatocyte growth factor gene (Ad-HGF) was obtained by homologous recombination (See FIG. 2). The structure of that recombinant adenovirus is as follows:

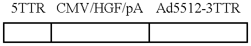

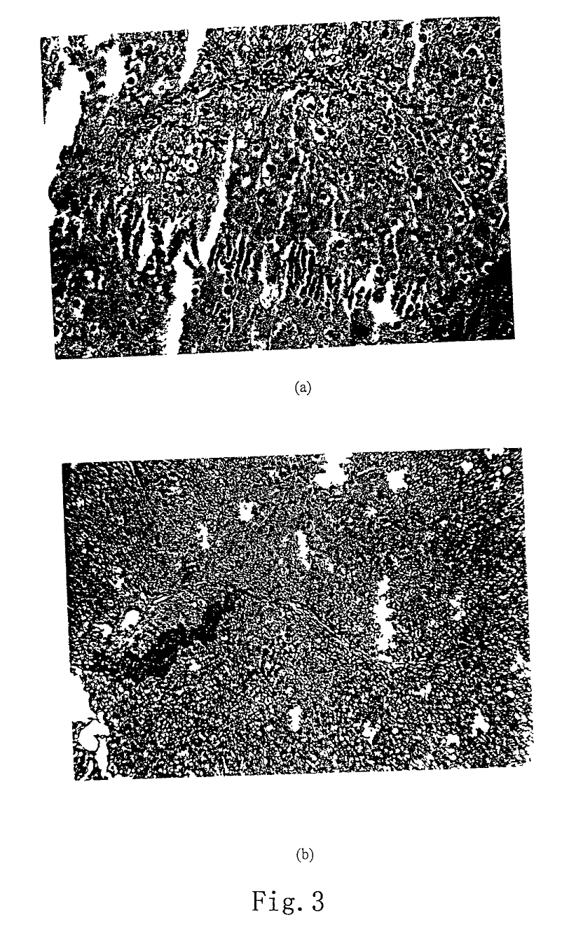

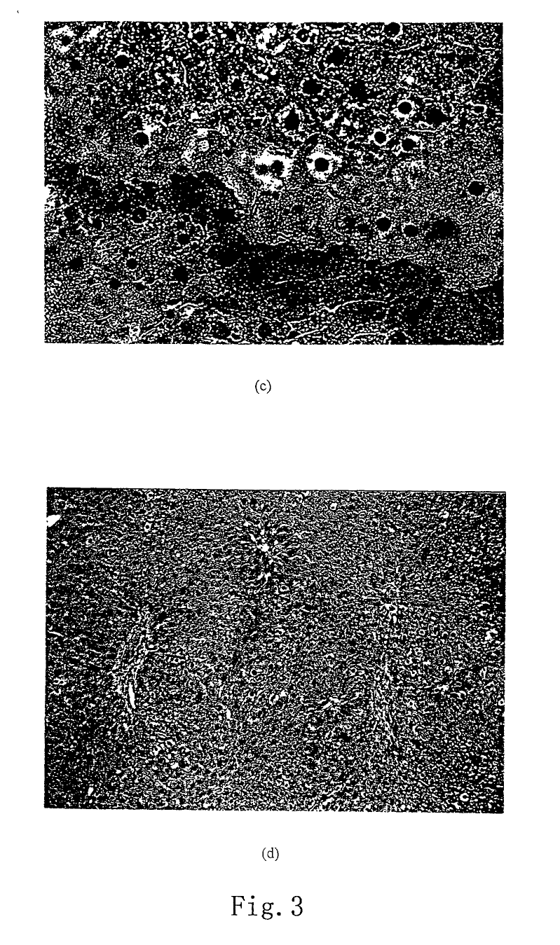

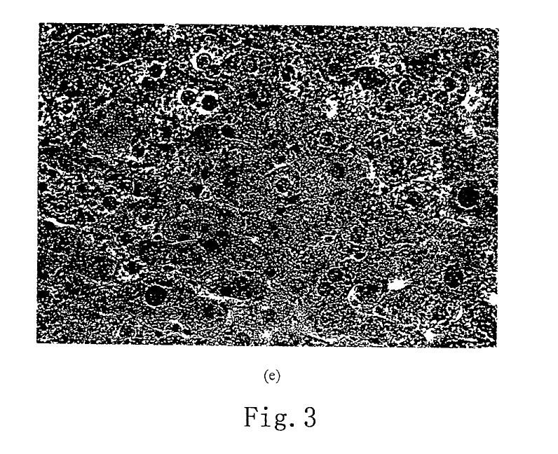

3. Injection of the obtained recombinant adenovirus into the caudal vein in a rat model, in which lethal liver fibrosis has been induced by dimetheylnitrosamine (DMN). Through histopathological observation, it was found that vacuolar degeneration in hepatocytes of the treatment group was milder in severity, the number of hepatocyte with that degeneration was fewer, and fibrous tissue around hepatic lobules was less than those of the control group (See FIGS. 3d,3e). Besides, psuedolobules were found in the control group (FIGS. 3a,3b,3c).Thus, the therapeutic effectiveness of the referred recombinant adenovirus was demonstrated.

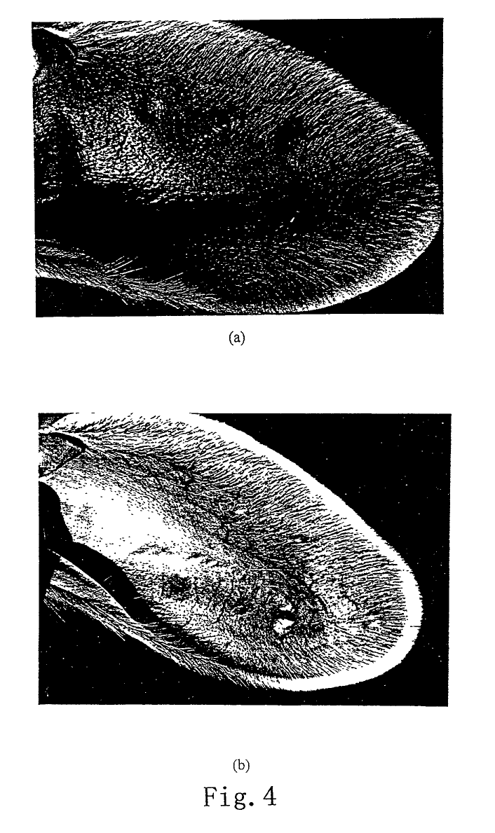

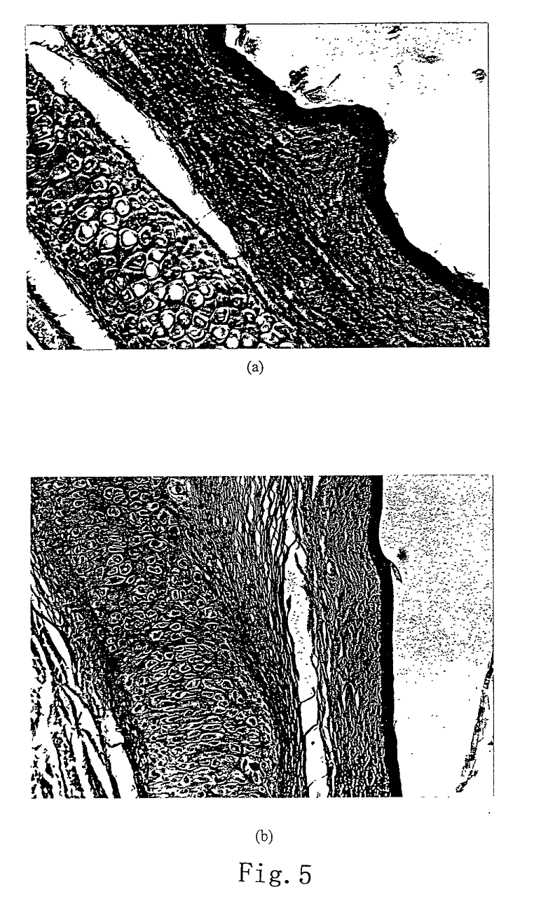

4. An ear scarring model was established by microsurgical technique in New Zealand white rabbits. After resection of ventral ear skin slips, a solution containing the referred recombinant adenovirus was immediately smeared on the surface of the fresh wound. The results demonstrated that wounds of the treatment group healed faster, inflammatory reaction was milder, and the scars were smaller(even without scar) than those of the control group (See FIGS. 4a,4b and FIGS. 5a,5b). Hence, it is demonstrated that the referred recombinant adenovirus could prevent from scar formation to a certain extent.

This invention provides a kind of recombinant adenovirus carrying human hepatocyte growth factor gene. The referred recombinant adenovirus can be used for preparation of liquid dosage forms (in normal saline or in water for injection), such as injections, smear solutions (for application on body surface),and so forth for prevention and treatment of fibrotic diseases. The types of fibrotic diseases can be liver fibrosis, scars, or fibrosis of other tissues and organs of the human body. The implementation of examples will expound this invention through describing gene therapy for liver fibrosis and skin scar formation using the referred recombinant adenovirus carrying human hepatocyte growth factor gene.

Application of the referred recombinant adenovirus provided by this invention in prevention and treatment of fibrotic diseases is based on the mechanism of pathogenesis of fibrous fibers and the high transfection efficiency and hepatophil nature of adenovirus. After induction of the human hepatocyte growth factor gene, the purpose of prevention and treatment of fibrotic diseases would be achieved through inhibition of synthesis of TGFβ and elevation of the activity of collagenase. Therefore, the means provided by this invention for prevention and treatment of fibrotic diseases using the referred recombinant adenovirus has sound scientific basis, more clear therapeutic effectiveness, and even reverse the process of fibrosis. Therefore, the subject matter as claimed in this invention may be as an effective mean for treatment of fibrotic diseases, for which there is no effective preventive and therapeutic means up to now.

A further description of this invention is as follows.

FIG. 1. A diagrammatic illustration of construction of the shuttle plasmid carrying human hepatocyte growth factor gene and the left-side sequence of adenovirus gene

FIG. 2. A diagrammatic illustration of the referred recombinant adenovirus carrying human hepatocyte growth factor gene

FIG. 3. Microscopic photographs showing the results of treatment of liver fibrosis using the referred recombinant adenovirus in rats (H.E. ,100×).

(a) (b) (c) control group (d) (e) treatment group

FIG. 4. Macroscopic photographs showing the results of prevention of scar formation using the referred recombinant adenovirus in rabbits.

(a) control group (b) prevention group

FIG. 5. Microscopic photographs showing the results of prevention of scar formation using the referred recombinant adenovirus in rabbits (H.E. ,100×).

(a) control group (b) prevention group

### REFERENCES

(1) Takahiro Ueki, Yasufumi Kaneda, Hiroko Tsutsui, et al. hepatocyte growth factor gene therapy of liver cirrhosis in rats. Nature Medicine, 1999;5(2):226

### EXAMPLES

I. Construction, amplification, purification of the referred recombinant adenovirus

1. Construction of the Referred Recombinant Adenovirus

FIG. 1 illustrated the construction steps of the shuttle plasmid carrying human hepatocyte growth factor gene (pXCJL1-CMV/HGF/pA). Cells of 293 cell line were co-infected with pXCJL1-CMV/HGF/pA and the recombinant plasmid GT4050 carrying adenoviral genome, which were recombined homologously within the cell, resulting in formation of the the referred recombinant adenovirus carrying human hepatocyte growth factor gene (named as Ad-HGF). The structure of Ad-HGF is shown schematically in FIG. 2.

2. Preparation and Purification of the Adenovirus

Cells of 293 cell line were inoculated in cell culture flasks. Adenovirus was added at 10 pfu/cell into the culture medium when 90% of the cultured cell grew confluently. After 36-48 hrs, when complete cytopathic effect appeared, the cells were harvested and stored at −80° C. As soon as cells in 30-40 flasks were cumulatively cultured, purification was carried out for one session. Repeated freezing at −80° C. and thawing at 37° C. of the 293 cells with cytopathic effect were performed for three times, then the cultured cells were centrifuged at 2500 rpm for 10 min and the supernatant was collected . A cesium chloride gradient was established: 0.5 ml of 1.5 g/ml cesium chloride, 2.5 ml of 1.35 g/ml cerium chloride, and 2.5 ml of 1.25 g/ml cesium chloride solutions in sterile PBS were added sequentially into ultracentrifugation tubes. The supernatant to be purified was added on the top of the cesium chloride gradient liquid at 6 ml/tube. Then centrifugation was carried out at 35000 rpm, 10° C. for one hour. A white, cloudy band of virus appeared between the 1.35 g/ml cesium chloride solution and 1.25 g/ml cesium chloride solution. The band of virus was collected, mixed with 1.35 g/ml cesium chloride solution, and centrifuged at 35000 rpm, 10° C. for 18 hours. Then the viral band was aspirated out and diluted with 2-fold volume of Hanks solution. Dialysis of the virus was carried out at 4° C. by taking Hanks solution as the dialyzate, which was changed every 2 hours, for 5 times. The purified viral liquid was taken out and sterile glycerin was added to final concentration of 10%. After divided into small parts, the virus was stored at −80° C. The titer of the virus was determined by plaque assay as 8.6×10¹¹ pfu/ml.

II. Evaluation of the therapeutic effectiveness of human hepatocyte growth factor gene mediated by recombinant adenovirus for treatment of liver fibrosis of rats

1. Establishment of the rat liver fibrosis model

Thirty male Wistar rats with body weight 200-250 g used. Among them 26 were randomly chosen for use in that model. One gram of dimethylnitrosamine (DMN) was dissolved in 100 ml sterile normal saline to a final concentration of 1%. That solution was injected subcutaneously to the 26 rats at 1 ml/kg at each time. The injection was given to the rats twice a day for 4 successive weeks. The rest 4 rats were served as the normal group, which were raised in the same condition as the model rats, except no DMN was given. On the tenth day after the last injection, 2 rats each were chosen randomly and sacrificed for macroscopic observation and preparation of routine histopathological slides of the liver for microscopic observation of the changes in fibrosis. Macroscopically the liver of the model group swelled remarkably with blunt edges and hard texture. By light microscopy, there were a large number of hepatocytes with evident vacuolar degeneration and widespreadly necrotic hepatocytes were seen. There were also infiltration by neutrophils and lymphocytes. In the portal area quite a lot of fibroblasts with a large quantity of fibrous tissue were present , which extended out to the periphery of the lobules, even encircling it in some fields. Hemorrhage was also found. In the liver of the normal rats none of the above-mentioned pathologic changes was noted. Hence, established of the model of liver fibrosis was achieved.

2. Treatment effect of the referred recombinant adenovirus on liver fibrosis

Twelve model rats were chosen and were randomly alloted to a treatment group and a control group. In the treatment group, 100 μl of Ad-HGF with 8.6×10⁷ pfu/μl were injected and repeated the injection once on the next day, whereas in the control group, same titers of Ad-GFP (recombinant adenovirus does not carry human hepatocyte growth factor gene but a gene encoding green fluorescent protein)were injected. On day 40 after treatment, the liver of rats of both groups were removed for preparation of routine histopathological slides. In the treatment group vacuolar degeneration of hepatocyte was milder and the number of hepatocytes with that change was fewer as compared with those of the control group. Besides, there were only scanty infiltrating lymphocytes and no neutrophil was seen. There was no hemorrhage. In the portal area the number of fibroblasts was fewer and only a scanty amount of collagenous fiber was seen. Fibrous tissue extending out from the portal area to the periphery of lobules was not evident, to say nothing of its encirelement of the lobules. In a part of the rat liver, all of the above-mentioned changes were even milder. On the contrary, all of the pathologic changes were remained as before in rats of the Ad-GFP group. Therefore, the therapeutic effectiveness was basically achieved.

III. Preventive effect of the referred recombinant adenovirus carrying human hepatocyte growth factor gene on scar formation

1. The animal model of scar formation

Areas without visible blood vessels of the ventral skin of ears of New Zealand rabbits (weighing 2.5 kg-3.5 kg) were shaved and disinfected, and circular whole-layered skin slips with a diameter of 0.6 cm were resected down to the top of the underlying cartilage with microsurgical technique. For one ear 5 slips were removed and both sides of the ears were used.

2. Evaluation of therapeutic effectiveness

Immediately after resection of skin slips and bleeding was stopped, a solution containing the referred recombinant adenovirus was smeared on the fresh surface of the wound of the treatment (Ad-HGF) group with 8 μl (8.6×10⁷ pfu/μl ). Then the smearing was repeated daily for 3 successive days. In the control (Ad-GFP) group the same amount of Ad-GFP was applied at the same time points. On day 22 the tissue of wound area, together with a brim of normal skin at its peripheral were resected for preparation of routine histopathological slides. By light microscopy, it was demonstrated that in the Ad-HGF group wounds healed faster, inflammatory reaction was milder, and scars were smaller as compared with those of the Ad-GFP group (See FIG. 4). Moreover, in the Ad-HGF group the epithelial cornification was milder, the thickness of the dermal layer was thinner and the amount of collagenous fiber was scanter than those of the Ad-GFP group (See FIG. 5). Hence, the preventive effect of the referred recombinant adenovirus on wound scar formation was demonstrated.

1. A recombinant adenovirus for the prevention and treatment of fibrosis, which characterises transducing the whole length cDNA of the domain encoding human hepatocyte growth factor into the referred recombinant adenovirus, the structure of the referred recombinant adenovirus is: 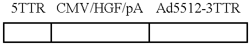
and the construction steps are shown in FIG. 1 and FIG. 2

2. A recombinant adenovirus according to claim 1, wherein said recombinant adenovirus is a type 5 replication-defective, E1 and E3 domains lacking adenovirus, which carrying the human hepatocyte growth factor gene.

3. A recombinant adenovirus according to claim 1, wherein said recombinant adenovirus is a kind of similar adenovirus (such as gutless adenovirus, etc) and other viral vectors (such as adeno-associated virus, etc) having the same function of carrying human hepatocyte growth factor as the adenovirus claimed in claim 2.

4. A recombinant adenovirus according to any one of claims 1-3, wherein said recombinant adenovirus is used in the form of injections, smear solutions, sprays, and other formulations containing a certain titer of the recombinant adenovirus for prevention and treatment of fibrotic diseases and vascular obliterative diseases.

5. A recombinant adenovirus according to claims 4, said fibrotic diseases is fibrosis of the liver, and the purpose of treatment of that disease is achieved by intravenous injection of solutions containing the referred recombinant adenovirus.

6. A recombinant adenovirus according to claims 4, wherein said fibrotic diseases is scars, the purpose of scars is achieved through smearing or spraying of solutions containing the referred recombinant adenovirus on the wound surface.

7. A recombinant adenovirus according to claims 4, wherein said fibrotic diseases is fibrotic diseases of any other organs of the human body (such as pulmonary fibrosis, and so on), the purpose of treatment of pulmonary fibrosis is achieved through inhalation of aerosol formulation containing the referred recombinant adenovirus.

8. A recombinant adenovirus according to claims 4, wherein said recombinant adenovirus is used for the treatment of vascular obliterative diseases, such as peripheral artery occlusive disease, obliterative disease of the coronary artery, etc.
